(19) United States
(12) Patent Application Publication
(10) Pub. No.: US 2022/0175649 A1
(43) Pub. Date: Jun. 9, 2022

(54) COMBINATION OF SPECIFIC PARTICLE AND LIPOPHILIC ANTIOXIDANT AGENT

(71) Applicant: L'OREAL, PARIS (FR)

(72) Inventors: Toru KOIKE, Kanagawa (JP); Yasuko HARADA, Kanagawa (JP)

(21) Appl. No.: 17/604,961

(22) PCT Filed: Apr. 3, 2020
(86) PCT No.: PCT/JP2020/016057

§ 371 Date: Oct. 19, 2021

(30) Foreign Application Priority Data
Apr. 26, 2019 (JP) 2019-085564

(51) Int. Cl. A61K 8/67; A61K 8/02; A61K 8/73

(52) U.S. Cl. CPC A61K 8/678; A61K 8/731; A61K 8/0279

(57) ABSTRACT

The present invention relates to a combination of: (a) at least one particle having a wet point for oil being at least 100 ml/100 g, preferably at least 150 ml/100 g, and more preferably at least 200 ml/100 g, and a wet point for water being at least 100 ml/100 g, preferably at least 200 ml/100 g, and more preferably at least 300 ml/100 g; and (b) at least one lipophilic antioxidant agent. The present invention also relates to a method or use characterized by using the above combination. The present invention can reduce or control more effectively, preferably synergistically, the peroxidation of unsaturated lipids, preferably sebum, compared to the single use of the lipophilic antioxidant agent.

### TECHNICAL FIELD

The present invention relates to a composition which is suitable for protecting keratin materials such as skin.

### BACKGROUND ART

The lipids occurring at the surface of the skin, scalp and hair are known to be continuously subjected to damaging external agents, in particular, atmospheric pollutants.

These lipids are those which form part of the constituents of the skin or hair, as well as those which are secreted by the skin including the scalp, and/or those which are deposited on the skin or hair when products containing lipids are applied to the skin or hair.

The lipids most exposed to damaging external agents are those contained in the fatty secretions of the skin such as sebum, which is rich in squalene. The presence of six double bonds in squalene makes squalene sensitive to oxidation. Thus, after prolonged exposure to pollutants in the air, squalene is peroxidized to give squalene peroxides.

This high production of squalene peroxides causes, in particular, a series of sequential degradations especially in and on the skin, giving rise to many skin disorders. Thus, these squalene peroxides participate in:
the pathogenesis of acne, as described by Saint Leger et al. (British Journal of Dermatology, vol. 114, pp. 535-542 (1986)), who point out that squalene peroxides are comedogenic;
premature skin aging, as described by Keiko Ohsawa et al. (The Journal of Toxicology Sciences, vol. 19, pp. 151-159 (1984)), who discuss the consequences of sun-induced skin burns;
irritation phenomena, as reported by Takayoshi Tanaka et al. (J. Clin. Biochem. Nutr., vol. 1, pp. 201-207 (1986)), who draw attention to the damage caused, in particular, by the repeated use of some shampoos;
the production of malodorous volatile products (aldehydes, ketones, acids, and the like); and
immunosuppression of biochemical messengers of the biological effects of UV irradiation of the skin, as described by M. Picardo et al. (Photodermatol. Photoimmunol. Photomed., vol. 3, pp. 105-110 (1991)).

In order to limit the peroxidation of unsaturated lipids, it is known to use an antioxidant agent. There are two classes of antioxidant agents. One is a hydrophilic antioxidant agent such as ascorbic acid or Vitamin C. The other is a lipophilic antioxidant agent such as tocopherol or Vitamin E.

### DISCLOSURE OF INVENTION

There has been a need for a new approach which is more effective for reducing or controlling the peroxidation of unsaturated lipids than the single use of an antioxidant agent.

Thus, an objective of the present invention is to provide a new approach which is more effective for reducing or controlling the peroxidation of unsaturated lipids.

The above objective can be achieved by a composition, preferably a cosmetic composition, and more preferably a cosmetic composition for protecting keratin materials such as skin, comprising:
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent.

The number-average primary particle size of the (a) particle may be 50 μm or less, preferably 30 μm or less, and more preferably 10 μm or less.

The ratio of the wet point for water/the wet point for oil of the (a) particle may be 5 or less, preferably 4 or less, and more preferably 2 or less.

It is preferable that the (a) particle be porous.

The (a) particle may comprise at least one material selected from the group consisting of polysaccharides, silicon compounds, boron compounds, metal compounds, polymers, perlites, and mixtures thereof.

It is preferable that the (a) particle comprise at least one polysaccharide, preferably cellulose.

The amount of the (a) particle in the composition according to the present invention may be from 0.01% to 20% by weight, preferably from 0.1% to 15% by weight, and more preferably from 1.0% to 10% by weight relative to the total weight of the composition.

The (b) lipophilic antioxidant agent may be selected from the group consisting of pentaerythrityl tetra-di-t-butyl hydroxyhydrocinnamate, nordihydroguaiaretic acid, propyl gallate, butylated hydroxytoluene, butylated hydroxyanisole, ascorbyl palmitate, tocopherol and mixtures thereof.

The (b) lipophilic antioxidant agent may be at least one tocopherol selected from the group consisting of α-tocopherol, β-tocopherol, γ-tocopherol, δ-tocopherol and a mixture thereof.

The amount of the (b) lipophilic antioxidant agent in the composition according to the present invention may be from 0.001% to 5% by weight, preferably from 0.005% to 1% by weight, and more preferably from 0.01% to 0.1% by weight relative to the total weight of the composition.

The composition according to the present invention may further comprise (c) at least one oil or (d) water. On the other hand, the composition according to the present invention may further comprise (c) at least one oil and (d) water. In the latter case, the composition according to the present invention may be in the form of a two-phase or multi-phase formulation, a W/O emulsion or an O/W emulsion, preferably in the form of a two-phase or a multi-phase formulation.

The composition according to the present invention may be intended for protecting skin from damage selected from the group consisting of oily skin, dehydration of skin, alteration of desquamation, squalene decrease, vitamin E decrease, pigmentation, pore problems such as clogged pores, dilated pores, acne and black heads, loss of dry/oily balance, dull skin, aging, and lactic acid increase.

The present invention also relates to a non-therapeutic method, preferably a cosmetic method, and more preferably a cosmetic method for protecting keratin materials such as skin, comprising:

applying onto the keratin materials a composition comprising:
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent.

The present invention also relates to a use of a combination of
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent

for reducing or controlling oxidation of unsaturated lipids.

### BEST MODE FOR CARRYING OUT THE INVENTION

After diligent research, the inventors have discovered a new approach which is more effective for reducing or controlling the peroxidation of unsaturated lipids.

The new approach is a combination of:
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent.

The (a) particle is amphiphilic, and the surface thereof may be hydrophilic, while the inner space thereof may be hydrophilic.

It is known that pollutants such as polycyclic aromatic hydrocarbons (PAHs) in the air can cause peroxidation of unsaturated lipids such as sebum. The (a) particle can absorb such pollutants or take such pollutants into the inner space thereof. Due to this entrapping of pollutants, the (a) particle can inhibit the pollutant from causing peroxidation of unsaturated lipids.

Without being bound by theory, it is assumed that the (a) particle can also absorb the (b) lipophilic antioxidant agent or take the (b) lipophilic antioxidant agent into the inner space thereof. Thus, the (b) lipophilic antioxidant agent can effectively exert anti-oxidation effects against the pollutants, preferably in the inner space of the (a) particle. This cooperation of the (a) particle and the (b) lipophilic antioxidant agent can provide improved anti-oxidation effects, preferably synergistic anti-oxidation effects.

Accordingly, the present invention can reduce or control the peroxidation of unsaturated lipids more effectively.

One of the aspects of the new approach is a composition, comprising:
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent.

The combination of the (a) particle with the above specific properties and the (b) lipophilic antioxidant agent can provide surprisingly improved effects, preferably synergistic effects, for reducing or controlling the peroxidation of unsaturated lipids compared to the single use of the (b) lipophilic antioxidant agent. Therefore, the present invention can reduce or control more effectively, preferably synergistically, the peroxidation of unsaturated lipids, preferably sebum, than the single use of the (b) lipophilic antioxidant agent.

Thus, the present invention can be useful for protecting skin from damage caused by the peroxidation of unsaturated lipids such as sebum, such as alteration of desquamation, squalene decrease, acne and black heads, dull skin, and aging, such as the formation of wrinkles and/or fine lines.

In addition, since the (a) particle can absorb pollutants or take such pollutants into the inner space thereof, the present invention can prevent or reduce a variety of damage caused by the pollutants, such as oily skin, dehydration of skin, alteration of desquamation, squalene decrease, vitamin E decrease, pigmentation, pore problems such as clogged pores, dilated pores, acne and black heads, loss of dry/oily balance, dull skin, aging, and lactic acid increase.

Another aspect of the new approach is a method or process characterized by using the above combination of the (a) particle with the above specific properties and the (b) lipophilic antioxidant agent.

The other aspect of the new approach is a use characterized by the above combination of the (a) particle with the above specific properties and the (b) lipophilic antioxidant agent.

Thus, the present invention relates to a composition, a method and a use, characterized by the above combination of the (a) particle with the above specific properties and the (b) lipophilic antioxidant agent.

Hereafter, each of the present inventions will be described in a detailed manner.

[Composition]

The composition according to the present invention comprises:
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent.

(Particle)

The composition according to the present invention includes (a) at least one particle with specific properties. If two or more particles are used, they may be the same or different.

The (a) particle used for the present invention has

a wet point for oil being at least 100 ml/100 g, preferably at least 150 ml/100 g, more preferably at least 200 ml/100 g, even more preferably at least 250 ml/100 g, and preferably 1500 ml/100 g or less; and

a wet point for water being at least 100 ml/100 g, preferably at least 200 ml/100 g, more preferably at least 300 ml/100 g, even more preferably at least 350 ml/100 g, and preferably 1500 ml/100 g or less.

The term “wet point for oil” in the specification means a quantity or amount of oil which is necessary to make a target powder completely wet, which can be recognized, in particular, by the formation of a paste with the target powder.

The wet point for oil can be determined by the following protocol.
(1) 2 g of a target powder is kneaded with a spatula on a glass plate while adding oil, in particular linear ester oil, such as isononyl isononanoate (WICKENOL 151/ALZO).
(2) When the target powder becomes completely wet and starts to form a paste, the weight of the added oil is determined as the weight of the wet point.
(3) The wet point for oil is calculated from the equation: Wet point for oil (ml/100 g)={(the weight of the wet point)/2 g}×100/the density of oil.

Similarly, the term “wet point for water” in the specification means a quantity or amount of water which is necessary to make a target powder completely wet, which can be recognized, in particular, by the formation of a paste with the target powder.

The wet point for water can be determined by the following protocol.
(1) 2 g of a target powder is kneaded with a spatula on a glass plate while adding water with a density of 0.998 g/ml.
(2) When the target powder becomes completely wet and starts to form a paste, the weight of the added water is determined as the weight of the wet point.
(3) The wet point for water is calculated from the equation: Wet point for water (ml/100 g)={(the weight of the wet point)/2 g}×100/the density of water.

It is preferable that the ratio of the wet point for water/the wet point for oil of the (a) particle used for the present invention be 5 or less, preferably 4 or less, more preferably 3 or less, and even more preferably 2 or less, and preferably 0.1 or more.

The particle size of the (a) particle used for the present invention is not limited. However, it is preferable that the number-average primary particle size of the (a) particle be 50 μm or less, preferably 30 μm or less, more preferably 10 μm or less, and even more preferably from 2 to 5 μm.

It is preferable that 90 vol % or more of the (a) particles used for the present invention have a number-average primary particle size ranging from 0.1 to 10 μm, preferably from 0.5 to 8 μm, and more preferably from 1 to 7 μm. If 90 vol % or more of the (a) particles have a number-average primary particle size ranging from 1 to 7 μm, optical effects due to the particles may also be achieved.

The number-average primary particle size can be measured by, for example, extracting and measuring from a photographic image obtained by SEM and the like, or using a particle size analyzer such as a laser diffraction particle size analyzer, and the like. It is preferable to use a particle size analyzer such as a laser diffraction particle size analyzer.

It is preferable that the ratio of the longest diameter/the shortest diameter of the (a) particle used for the present invention range from 1.0 to 10, preferably from 1.0 to 5, and more preferably from 1.0 to 3.

The (a) particle used for the present invention may be porous or non-porous. It is preferable, however, that the (a) particle used in the present invention be porous.

The porosity of the (a) particle may be characterized by a specific surface area of from 0.05 m²/g to 1,500 m²/g, more preferably from 0.1 m²/g to 1,000 m²/g, and even more preferably from 0.2 m²/g to 500 m²/g according to the BET method.

The (a) particle can comprise any materials, which are not limited to, and can be selected from polysaccharides such as cellulose; silicon compounds such as silica; boron compounds such as boron nitride; metal compounds such as alumina, barium sulfate and magnesium carbonate; polymers such as polyamide, especially nylon, acrylic polymers, especially of polymethyl methacrylate, of polymethyl methacrylate/ethylene glycol dimethacrylate, of polyallyl methacrylate/ethylene glycol dimethacrylate or of ethylene glycol dimethacrylate/lauryl methacrylate copolymer; perlites; and mixtures thereof.

It is preferable that the (a) particle comprises at least one material selected from the group consisting of polysaccharides, silicon compounds, boron compounds, metal compounds, polymers, perlites, and mixtures thereof.

It is more preferable that the (a) particle comprises at least one polysaccharide.

The polysaccharide may be selected from alginic acid, guar gum, xanthan gum, gum arabic, arabinogalactan, carrageenan, agar, karaya gum, gum tragacanth, tara gum, pectin, locust bean gum, cardolan, gellan gum, dextran, pullulan, hyaluronic acid, cellulose and its derivatives, and mixtures thereof. Cellulose and its derivatives are preferable. Cellulose is more preferable.

In the present invention, the cellulose that may be used is not limited by the types of cellulose such as cellulose I, cellulose II, or the like. As the cellulose which can be used as a material for the (a) particle for the present invention, type II cellulose is preferable.

The cellulose which can be used as a material for the (a) particle in the composition used for the present invention may be in any particulate form, in particular, a spherical particle.

The cellulose particle, preferably a spherical cellulose particle, can be prepared, for example, as follows.
(1) A slurry of calcium carbonate, as an aggregation inhibitor, is added to an alkaline water-soluble anionic polymer aqueous solution, and stirred.
(2) Viscose and the aqueous solution obtained in the above (1) are mixed to form a dispersion of viscose fine particles.
(3) The dispersion of viscose fine particles obtained in the above (2) is heated to aggregate the viscose in the dispersion, and neutralized with acid, to form cellulose fine particles.
(4) The cellulose fine particles are separated from the mother liquid obtained in the above (3), and washed and dried, if necessary.

The viscose is a raw material of the cellulose. It is preferable to use viscose with a gamma value of 30 to 100% by mass and an alkaline concentration of 4 to 10% by mass. As the above water-soluble anionic polymer, mention may be made of polyacrylic acid sodium salt, polystyrene sulfonic acid sodium salt, and the like. The above calcium carbonate is used to prevent the aggregation of viscose fine particles in the dispersion and to make the particle size of the cellulose particle smaller. As the calcium carbonate slurry, mention may be made of Tama Pearl TP-221GS marketed by Okutama Kogyo Co., Ltd. in Japan.

According to one embodiment, a cellulose derivative may be chosen from cellulose esters and ethers.

It is indicated that the term “cellulose ester” means, in the text hereinabove and hereinbelow, a polymer consisting of an α (1-4) sequence of partially or totally esterified anhydroglucose rings, the esterification being obtained by the reaction of all or only some of the free hydroxyl functions of the said anhydroglucose rings with a linear or branched carboxylic acid or carboxylic acid derivative (acid chloride or acid anhydride) containing from 1 to 4 carbon atoms.

Preferably, the cellulose ester results from the reaction of some of the free hydroxyl functions of said rings with a carboxylic acid containing from 1 to 4 carbon atoms.

Advantageously, the cellulose esters are chosen from cellulose acetates, propionates, butyrates, isobutyrates, acetobutyrates and acetopropionates, and mixtures thereof.

These cellulose esters may have a weight-average molecular mass ranging from 3,000 to 1,000,000, preferably from 10,000 to 500,000 and more preferably from 15,000 to 300,000.

In the text hereinabove and hereinbelow, the term “cellulose ether” means a polymer consisting of an α (1-4) sequence of partially etherified anhydroglucose rings, some of the free hydroxyl functions of said rings being substituted with a radical —OR, R preferably being a linear or branched alkyl radical containing from 1 to 4 carbon atoms.

The cellulose ethers are thus preferably chosen from cellulose alkyl ethers with an alkyl group containing from 1 to 4 carbon atoms, such as cellulose methyl, propyl, isopropyl, butyl and isobutyl ethers.

These cellulose ethers may have a weight-average molecular mass ranging from 3,000 to 1,000,000, preferably from 10,000 to 500,000 and more preferably from 15,000 to 300,000.

As the (a) particle used for the present invention, mention may be made of, for example, the following spherical cellulose particles marketed by Daito Kasei in Japan:

Cellulobeads USF (the wet point for oil is 296.0 ml/100 g, the wet point for water is 400.8 ml/100 g, and the ratio of the wet point for water/the wet point for oil is 1.4) with a particle size of 4 μm (porous cellulose).

It is also preferable that the (a) particle used in the present invention comprises at least one silicon compound, preferably silicon oxide, and more preferably silica.

A silica suitable for the present invention is a hydrophilic silica selected from precipitated silicas, fumed silicas and mixtures thereof.

A silica suitable for the present invention may be spherical or non-spherical in shape, and may be porous or nonporous. In one of the embodiments of the present invention, a silica suitable for the present invention is spherical and porous. The porosity of a silica particle may be open to the exterior or in the form of a central cavity.

A silica may be hydrophilic.

It is also preferable that the (a) particle used in the present invention comprise boron nitride.

The most preferred form of boron nitride used for the powder in accordance with the present invention is hexagonal boron nitride. One suitable line of products is available as Combat® boron nitride powders, from Standard Oil Engineered Materials Company, Niagara Falls, N.Y.; high purity grades and specifically grade SHP3 are preferred.

The (a) particle used for the present invention may or may not be coated beforehand.

In a particular embodiment, the (a) particle is originally coated. The material of an original coating of the particle is not limited, but an organic material such as a mono- or di-carboxylic acid or a salt thereof, an amino acid, an N-acylamino acid, an amido, a silicone and a modified silicone may be preferable. As the organic material, mention may be made of potassium succinate, lauroyl lysine and acryl-modified silicone.

In other words, the (a) particle used for the present invention may be surface-treated. As examples of the surface treatments, mention may be made of the following:
(1) Fluorine-based compound treatments such as treatments with perfluoroalkylphosphates, perfluoroalkylsilanes, perfluoropolyethers, fluorosilicones, and fluorinated silicone resins
(2) Silicone treatments such as treatments with methylhydrogenpolysiloxanes, dimethylpolysiloxanes, and tetramethyltetrahydrogencyclotetrasiloxane in a gas phase
(3) Pendant treatments such as treatments to add an alkyl chain and the like after the gas phase silicone treatment
(4) Silane coupling agent treatments
(5) Titanium coupling agent treatments
(6) Aluminum coupling agent treatments
(7) Oil agent treatments
(8) N-acylated lysine treatments
(9) Polyacrylic acid treatments
(10) Metal soap treatments such as those with stearate salt or myristate salt
(11) Acrylic resin treatments
(12) Metal oxide treatments

It is possible to perform a plurality of surface treatments in combination with the above treatments.

As the (a) particle used for the present invention, Cellulobeads USF, Sunsphere H33 and Boron Nitride SHP3 are preferable. Cellulobeads USF and Sunsphere H33 are more preferable, and Cellulobeads USF is most preferable.

The amount of the (a) particle(s) in the composition according to the present invention may be from 0.01% by weight or more, preferably 0.1% by weight or more, and more preferably 1.0% by weight or more, relative to the total weight of the composition.

The amount of the (a) particle(s) in the composition according to the present invention may be 20% by weight or less, preferably 15% by weight or less, and more preferably 10% by weight or less, relative to the total weight of the composition.

The amount of the (a) particle(s) in the composition according to the present invention may be from 0.01% to 20% by weight, preferably from 0.1% to 15% by weight, and more preferably from 1.0% to 10% by weight relative to the total weight of the composition.

(Lipophilic Antioxidant Agent)

The composition according to the present invention includes (b) at least one lipophilic antioxidant agent. A single type of lipophilic antioxidant agent may be used, but two or more different types of lipophilic antioxidant agents may be used in combination.

According to the present invention, antioxidant agents are compounds or substances that can scavenge the various radical forms which may be present in the skin; preferably, they simultaneously scavenge all of the various radical forms present.

The (b) lipophilic antioxidant agent means that the partition coefficient of the antioxidant agent between n-butanol and water is >1, more preferably >10 and even more preferably >100.

The (b) lipophilic antioxidant agent is hydrophobic, and is not a hydrophilic antioxidant agent such as ascorbic acid or glutathione.

As the (b) lipophilic antioxidant agent, mention may be made of phenolic antioxidants which have a hindered phenol structure or a semi-hindered phenol structure within the molecule.

As specific examples of such compounds, mention may be made of 3,5-bis(1,1-dimethylethyl)-4-hydroxybenzenepropanoic acid) which has the INCI name of pentaerythrityl tetra-di-t-butyl hydroxyhydrocinnamate, 2,6-di-tert-butyl-4-methylphenol, 2,6-di-tert-butyl-4-ethylphenol, mono- or di- or tri-(α-methylbenzyl)phenol, 2,2′-methylenebis(4-ethyl-6-tert-butylphenol), 2,2′-methylenebis(4-methyl-6-tert-butylphenol), 4,4′-butylidenebis(3-methyl-6-tert-butylphenol), 4,4′-thiobis(3-methyl-6-tert-butylphenol), 2,5-di-tert-butylhydroquinone, 2,5-di-tert-amylhydroquinone, tris[N-(3,5-di-tert-butyl-4-hydroxybenzyl)]isocyanurate, 1,1,3-tris(2-methyl-4-hydroxy-5-tert-butylphenyl)butane, butylidene-1,1bis[3-(3-tert-butyl-4-hydroxy-5-methylphenyl)propionate], octadecyl 3-(3,5-di-tert-butyl-4-hydroxyphenyl)propionate, tetrakis[methylene-3-(3,5-di-tert-butyl-4-hydroxyphenyl)propionato]methane, triethylene glycol bis[3-(3-tert-butyl-4-hydroxy-5-methylphenyl)propionate], 3,9-bis{2-[3-(3-tert-butyl-4-hydroxy-5-methylphenyl)propionyloxy]-1,1-dimethylethyl}-2,4,8,10-tetraoxaspiro [5.5]undecane, 1,3,5-trimethyl-2,4,6-tris(3,5-di-tert-butyl-4-hydroxybenzyl)benzene, 2,2-thiodiethylenebis[3-(3,5-di-tert-butyl-4-hydroxyphenyl)propionate], N,N′-hexamethylenebis(3,5-di-tert-butyl-4-hydroxyhydrocinnamide), 1,6-hexanediol bis[3-(3,5-di-tert-butyl-4-hydroxyphenyl)propionate], 1,3,5-tris[(4-tert-butyl-3-hydroxy-2,6-xylyl)methyl]-1,3,5-triazine-2,4,6-trione, 2,4-bis(n-octylthio)-6-(4-hydroxy-3,5-di-tert-butylanilino)-1,3,5-triazine, 2-tert-butyl-6-(3′-tert-butyl-5′-methyl-2′-hydroxybenzyl)-4-methylphenyl acrylate, 2-[1-(2-hydroxy-3,5-di-tert-pentylphenyl)ethyl]-4,6-di-tert-pentylphenyl acrylate, 4,6-bis[(octylthio)methyl]-o-cresol, 2,4-di-tert-butylphenyl-3,5-di-tert-butyl-4-hydroxybenzoate and 1,6-hexanediolbis[3-(3,5-di-tert-butyl-4-hydroxyphenyl) propionate].

As the (b) lipophilic antioxidant agent, mention may be made of BHA (butylated hydroxyl anisole) and BHT (butylated hydroxyl toluene), vitamin E (or tocopherols and tocotrienol) and derivatives thereof, such as the phosphate derivative, for instance TPNA® sold by the company Showa Denko, coenzyme Q10 (or ubiquinone), idebenone, certain carotenoids such as lutein, astaxanthin, beta-carotene, polyphenols, phenolic acids and derivatives (e. g., chlorogenic acid), and flavonoids, which represent the main subgroup of polyphenols.

Among the flavonoids, mention may be made especially of chalcones, hydroxylated chalcones and reduced derivatives thereof (as described especially in patent FR 2 608 150), for instance phloretin, neohesperidin, phloridzin, aspalathin, etc., flavanones, for instance hesperetin and naringin, flavonols, for instance quercetin, rutin, flavanols, for instance catechin, EGCG, flavones, for instance apigenidin, and finally anthocyans. Mention may also be made of tannins. Reference may also be made to the compounds described in patent applications FR 2 699 818, FR 2 706 478, FR 2 907 339, FR 2 814 943 and FR 2 873 026.

Polyphenol compounds may especially be derived from plant extracts chosen from extracts of green tea, apple, hop, guava, cocoa, or wood such as chestnut, oak, horse chestnut or hazel. It is also possible to use an extract of pinaster bark, for example obtained according to processes described in U.S. Pat. Nos. 4,698,360, 6,372,266 and 5,720,956. As examples of such extracts, the compound referenced by the INCI name Pinus pinaster (bark extract) and by the CTFA name pine (Pinus pinaster) bark extract may be cited. It may, in particular, be the extract of pinaster bark marketed under the name PYCNOGENOL® by the BIOLANDES AROMES firm and/or HORPHAG Research. The extracts of (Maritime) pine bark from LAYN Natural Ingredients, Pine Bark from Blue California, and also Oligopin® from D.R.T. (Les Derives Resiniques et Terpeniques) may also be cited.

In the context of the present invention, the term “polyphenol compound” thus also covers the plant extract itself, rich in these polyphenol compounds.

The (b) lipophilic antioxidant agents that may also be mentioned include dithiolanes, for instance asparagusic acid, or derivatives thereof, for instance siliceous dithiolane derivatives, especially such as those described in patent application FR 2 908 769.

The (b) lipophilic antioxidant agents that may also be mentioned include:

glutathione and derivatives thereof (GSH and/or GSHOEt), such as glutathione alkyl esters (such as those described in patent applications FR 2 704 754 and FR 2 908 769); and

cysteine and derivatives thereof, such as N-acetylcysteine or L-2-oxothiazolidine-4-carboxylic acid. Reference may also be made to the cysteine derivatives described in patent applications FR 2 877 004 and FR 2 854 160; and

ferulic acid and derivatives thereof (esters, salts, etc.). Mention may particularly be made of esters of ferulic acid and of C1-C30 alcohols, in particular methyl ferulate, ethyl ferulate, isopropyl ferulate, octyl ferulate and oryzanyl ferulate;

certain enzymes for defending against oxidative stress, such as catalase, superoxide dismutase (SOD), lactoperoxidase, glutathione peroxidase and quinone reductases;

benzylcyclanones; substituted naphthalenones; pidolates (as described especially in patent application EP 0 511 118); caffeic acid and derivatives thereof, gamma-oryzanol; melatonin, sulforaphane and extracts containing it (excluding cress);

the diisopropyl ester of N,N′-bis(benzyl)ethylenediamine-N,N′-diacetic acid, as described especially in patent applications WO 94/11338, FR 2 698 095, FR 2 737 205 or EP 0 755 925; and

deferoxamine (or desferal) as described in patent application FR 2 825 920.

The (b) lipophilic antioxidant agents that are preferably used are the chalcones, more particularly phloretin or neohesperidin, the diisopropyl ester of N,N′-bis(benzyl)ethylenediamine-N,N′-diacetic acid, or an extract of pinaster bark such as PYCNOGENOL®.

As examples of the (b) lipophilic antioxidant agent, mention may be made of pentaerythrityl tetra-di-t-butyl hydroxyhydrocinnamate, nordihydroguaiaretic acid, propyl gallate, butylated hydroxyl toluene, butylated hydroxyl anisole, ascorbyl palmitate, tocopherol, and mixtures thereof.

The (b) lipophilic antioxidant agent may be at least one tocopherol selected from the group consisting of α-tocopherol, β-tocopherol, γ-tocopherol, δ-tocopherol and a mixture thereof. Vitamin E may also be used as the (b) lipophilic antioxidant agent.

The amount of the (b) lipophilic antioxidant agent(s) in the composition according to the present invention may be from 0.001% by weight or more, preferably 0.005% by weight or more, and more preferably 0.01% by weight or more, relative to the total weight of the composition.

The amount of the (b) lipophilic antioxidant agent(s) in the composition according to the present invention may be 5% by weight or less, preferably 1% by weight or less, and more preferably 0.1% by weight or less, relative to the total weight of the composition.

The amount of the (b) lipophilic antioxidant agent(s) in the composition according to the present invention may be from 0.001% to 5% by weight, preferably from 0.005% to 1% by weight, and more preferably from 0.01% to 0.1% by weight relative to the total weight of the composition.

(Oil)

The composition according to the present invention may comprise (c) at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

It is preferable that the oil be selected from synthetic oils, hydrocarbon oils, and mixtures thereof, more preferably from ester oils, hydrocarbon oils and mixtures thereof, and even more preferably from ester oils.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; and diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold, in particular, under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of a type such as dimethylsiloxane/methylalkylsiloxane, such as Silicone Volatile® FZ 3109 sold by the company Union Carbide, of the formula:

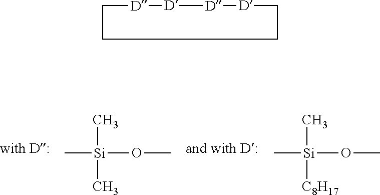

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold, in particular, under the name SH 200 by the company Toray Silicone. Silicones belonging to this category are also described in the article published in Cosmetics and Toiletries, Vol. 91, January '76, pp. 27-32, Todd & Byers, Volatile Silicone Fluids for Cosmetics. The viscosity of the silicones is measured at 25° C. according to ASTM standard 445, Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

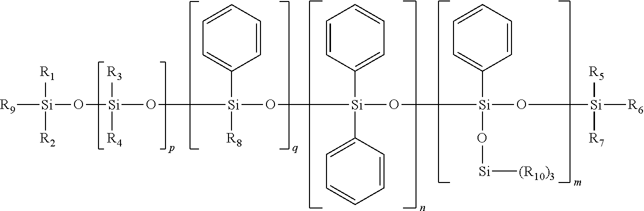

in which

R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and

m, n, p and q are, independently of each other, integers from 0 to 900 inclusive, preferably 0 to 500 inclusive, and more preferably 0 to 100 inclusive,

with the proviso that the sum of n+m+q is something other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; and m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from octyldodecanol, hexyldecanol and mixtures thereof.

It is may be preferable that the (c) oil is chosen from polar oils, more preferably ester oils. In other words, it may be preferable that the (c) oil comprises at least one polar oil, and more preferably at least one ester oil.

The amount of the (c) oil(s) in the composition according to the present invention may be from 1% by weight or more, preferably 3% by weight or more, and more preferably 5% by weight or more, relative to the total weight of the composition.

The amount of the (c) oil(s) in the composition according to the present invention may be 50% by weight or less, preferably 45% by weight or less, and more preferably 40% by weight or less, relative to the total weight of the composition.

The amount of the (c) oil(s) in the composition according to the present invention may be from 1% to 50% by weight, preferably from 3% to 45% by weight, and more preferably from 5% to 40% by weight relative to the total weight of the composition.

(Water)

The composition according to the present invention may comprise (d) water.

The amount of the (d) water in the composition according to the present invention may be from 10% by weight or more, preferably 20% by weight or more, and more preferably 30% by weight or more, relative to the total weight of the composition.

The amount of the (d) water in the composition according to the present invention may be 95% by weight or less, preferably 90% by weight or less, and more preferably 85% by weight or less, relative to the total weight of the composition.

The amount of the (d) water in the composition according to the present invention may be from 10% to 95% by weight, preferably from 20% to 90% by weight, and more preferably from 30% to 85% by weight relative to the total weight of the composition.

(Additional Optional Ingredient)

The composition according to the present invention may also include at least one additional optional ingredient.

The additional optional ingredient may be selected from the group consisting of cationic, anionic, nonionic, or amphoteric polymers; anionic, nonionic, or amphoteric surfactants; organic or inorganic UV filters; peptides and derivatives thereof; protein hydrolyzates; swelling agents and penetrating agents; agents for combating hair loss; anti-dandruff agents; natural or synthetic thickeners; suspending agents; sequestering agents; opacifying agents; dyes; sunscreen agents; vitamins or provitamins other than the ingredient (b); fragrances; preservatives, co-preservatives, stabilizers; and mixtures thereof.

The amount of the additional optional ingredient is not limited, but may be from 0.01% to 30% by weight, preferably from 0.1% to 20% by weight, and more preferably from 1% to 10% by weight, relative to the total weight of the composition according to the present invention.

[Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

[Form]

The composition according to the present invention may be in various forms.

If the composition according to the present invention includes (c) at least one oil or (d) water, the composition according to the present invention may be, for example, in the form of a suspension, a dispersion, or a solution. The aspect of the composition according to the present invention may be a fluid, a gel, a paste or a cream.

If the composition according to the present invention includes (c) at least one oil and (d) water, the composition according to the present invention may be, for example, in the form of emulsions such as oil-in-water (O/W), water-in-oil (W/O), and multiple (e.g., W/O/W, polyol/O/W, and O/W/O) emulsions, or two-phase or multi-phase formulations. It may be preferable that the composition according to the present invention be in the form of a two-phase or multi-phase formulation or a W/O emulsion, more preferably in the form of a two-phase or a multi-phase formulation. In the multi-phase formulation, the (a) particle may form a powder phase, the (c) oil may form an oil phase, and the (d) water may form an aqueous phase. The aspect of the composition according to the present invention may be a fluid, a gel, a paste or a cream.

[Cosmetic Composition]

The composition according to the present invention may be a cosmetic composition, preferably a cosmetic composition for protecting keratin materials such as skin, and more preferably a cosmetic composition for protecting the face.

The keratin materials may be selected from the group consisting of the skin, scalp, lips and hair.

It is preferable that the composition according to the present invention be used as a skin cosmetic composition.

The composition according to the present invention may be intended for protecting skin from damage selected from the group consisting of oily skin, dehydration of skin, alteration of desquamation, squalene decrease, vitamin E decrease, pigmentation, pore problems such as clogged pores, dilated pores, acne and black heads, loss of dry/oily balance, dull skin, aging, and lactic acid increase.

Preferably, the composition according to the present invention may be used for protecting skin from alteration of desquamation, squalene decrease, acne and black heads, dull skin, and aging such as the formation of wrinkles and/or fine lines.

The composition used for the present invention may be in the form of, for example, a lotion or a cosmetic water, a serum, a milk, a cream, a base foundation, an undercoat, a make-up base coat, a foundation, a lipstick, a lip cream, an eye shadow, an eyeliner, a concealer, a nail coat, a mascara, a sunscreen, a cleanser, and the like.

It is to be understood that a person skilled in the art can choose the appropriate presentation form, as well as its method of preparation, on the basis of his/her general knowledge, taking into account the nature of the constituents used, for example, their solubility in the vehicle, and the application envisaged for the composition.

[Method]

The present invention also relates to a non-therapeutic method or process, preferably a cosmetic method or process, and more preferably a cosmetic method or process for protecting keratin materials such as skin, comprising:

applying onto the keratin materials a composition comprising:
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent.

The keratin materials may be selected from the group consisting of the skin, scalp, lips and hair.

Urban environments are regularly subjected to peaks of pollution. An individual in his daily environment, and particularly in an urban zone, may be subjected to a whole range of factors attacking keratin materials, and in particular the skin, the scalp and the hair, by various airborne pollutants. Atmospheric pollutants which are represented largely by the primary and secondary products of combustion represent a major source of environmental oxidative stress. Urban pollution is composed of various types of chemical and xenobiotic products and particles. The major categories of pollutants which may exert harmful effects on the skin and the hair are as follows: gases, heavy metals, polycyclic aromatic hydrocarbons (PAHs) and particulate elements which are combustion residues onto which are adsorbed a very large number of organic and mineral compounds.

It is the outermost tissues that are initially and directly exposed to environmental toxins. The skin is directly and frequently exposed to a pro-oxidative environment and it is particularly sensitive to the action of oxidative stress; its outermost layer serves as a barrier to oxidative damage which may take place. In the majority of circumstances, the oxidizing agent is generally neutralized after reaction with the keratin materials, but the reaction products formed may be responsible for attacks on cells and tissues. The stratum corneum, the skin's barrier, is the site of contact between the air and skin tissue, and the lipid/protein two-phase structure is a crucial factor of this barrier function of the skin. These elements may react with the oxidizing agents and become impaired, which will promote the desquamation phenomena.

Among the pollutants that may exert deleterious effects on keratin materials, toxic gases such as ozone, carbon monoxide, nitrogen oxides or sulphur oxides are among the major constituents of pollutants. It has been found that these toxic gases promote the desquamation of keratin materials; they “fatigue” the keratin materials, that is to say make them dull and dirty. Similarly, cellular asphyxia of the keratin materials has been observed.

It is known that heavy metals (lead, cadmium and mercury) are atmospheric pollutants whose emissions have increased considerably, especially in urban and industrial environments. Although the majority of the effects of these metals are seen in other tissues (lungs, kidneys, brain, etc.), it has been shown that certain metals can penetrate into the skin and become accumulated therein (A. B. G. Landsdown, Critical Reviews in Toxicology, 1995, Vol. 25, pp. 397462).

In addition to certain toxic effects which they cause, heavy metals have the property of reducing the activity of the cellular defense means against free radicals [see for example R. S. Dwivedi, J. Toxicol. Cut. & Ocular Toxical. 6(3), 183-191 (1987)). Thus, heavy metals aggravate the toxic effects of gaseous pollutants by reducing the efficacy of the natural defense means, and bring about an acceleration of the phenomenon of cell ageing. This is particularly true for keratin materials and especially the skin, the scalp and the hair, which are in direct and permanent contact with the external environment.

Another major category of pollutants consists of combustion residues in the form of particles onto which are adsorbed a very large number of organic compounds, and in particular of polycyclic aromatic hydrocarbons (PAHs) such as benzopyrene. These PAHs adsorbed at the surface of the particles and dust borne by the urban atmosphere can penetrate into skin tissue and become stored and/or biotransformed therein.

Thus, the harmful effects of pollution on keratin materials affect cell respiration and are reflected by accelerated ageing of the skin, with a dull complexion and the early formation of wrinkles or fine lines, and also by a reduction in the vigour of the hair, which thus acquires a dull appearance. In addition, due to pollution, the skin and hair become dirty more quickly.

The present invention can combat the damage caused by the pollutants. Examples of such damage include oily skin, dehydration of skin, alteration of desquamation, squalene decrease, vitamin E decrease, pigmentation, pore problems such as clogged pores, dilated pores, acne and black heads, loss of dry/oily balance, dull skin, skin aging, and lactic acid increase.

Thus, the method according to the present invention can be used for protecting skin from damage selected from the group consisting of oily skin, dehydration of skin, alteration of desquamation, squalene decrease, vitamin E decrease, pigmentation, pore problems such as clogged pores, dilated pores, acne and black heads, loss of dry/oily balance, dull skin, aging, and lactic acid increase.

In particular, sebum peroxidation is considered to be one of the causes of the damage to keratin materials such as skin. Examples of such damage include alteration of desquamation, squalene decrease, acne and black heads, dull skin, and aging such as the formation of wrinkles and/or fine lines.

Thus, preferably, the composition according to the present invention can be used for protecting skin from alteration of desquamation, squalene decrease, acne and black heads, dull skin, and aging such as the formation of wrinkles and/or fine lines.

[Use]

The present invention also relates to a use of a combination of
(a) at least one particle having
a wet point for oil being at least 100 ml/100 g, preferably at least 150 ml/100 g, and more preferably at least 200 ml/100 g, and
a wet point for water being at least 100 ml/100 g, preferably at least 200 ml/100 g, and more preferably at least 300 ml/100 g; and
(b) at least one lipophilic antioxidant agent

for reducing or controlling oxidation of unsaturated lipids.

Examples of unsaturated lipids include squalene. Sebum is rich in squalene. Thus, sebum is a typical example of an unsaturated lipid. Since sebum is a typical example of an unsaturated lipid, the use according to the present invention can be for reducing or controlling the oxidation of sebum, in particular the peroxidation of sebum.

The above combination of the (a) particle with the above specific properties and the (b) lipophilic antioxidant agent can provide surprisingly improved effects, preferably synergistic effects, for reducing or controlling the peroxidation of unsaturated lipids, such as sebum, compared to the single use of the (b) lipophilic antioxidant agent. Therefore, the use according to the present invention can reduce or control more effectively, preferably synergistically, the peroxidation of unsaturated lipids, such as sebum, than the single use of the (b) lipophilic antioxidant agent.

### EXAMPLES

The present invention will be described in more detail by way of examples, which, however, should not be construed as limiting the scope of the present invention.

### Example 1 and Comparative Examples 1-4

[Preparation]

The following cosmetic compositions according to Example 1 and Comparative Examples 1-4, shown in Table 1, were prepared by mixing the ingredients shown in Table 1. The numerical values for the amounts of the ingredients shown in Table 1 are all based on “% by weight” as active raw materials.

TABLE 1

| | | Comp. | Comp. | Comp. | Comp. |
|---|---|---|---|---|---|
| | Ex. 1 | Ex. 1 | Ex. 2 | Ex. 3 | Ex. 4 |
| Form | Two-Phase | | | | |
| Disodium EDTA | 0.2 | 0.2 | 0.2 | 0.2 | 0.2 |
| Phenoxyethanol | 0.7 | 0.7 | 0.7 | 0.7 | 0.7 |
| Chlorphenesin | 0.15 | 0.15 | 0.15 | 0.15 | 0.15 |
| Ethanol | 5 | 5 | 5 | 5 | 5 |
| Water | qsp 100 | qsp 100 | qsp 100 | qsp 100 | qsp 100 |
| Glycerin | 3 | 3 | 3 | 3 | 3 |
| Isopropyl Myristate | 5 | 5 | 5 | 5 | 5 |
| Cellulose Beads | 2 | — | — | 2 | 2 |
| Tocopherol | 0.05 | — | 0.05 | — | — |
| Glutathione | — | — | — | 0.05 | — |
| Ascorbic Acid | — | — | — | — | 0.05 |
| Hydroperoxide | 4 | 20 | 10 | 9 | 8 |
| Concentration (μM) | | | | | |

The properties of the cellulose beads are shown in Table 2.

TABLE 2

| | | Mean Size | WP Oil | WP Water |
|---|---|---|---|---|
| Powder | Shape | (μm) | (ml/100 g) | (ml/100 g) |
| Cellulose Beads | Sphere | 4.0 | 296.0 | 400.8 |
| (Cellulobeads USF, | | | | |
| Daito Kasei) | | | | |

Mean Size: Number-Average Primary Particle Size
WP Oil: Wet Point for Oil
WP Water: Wet Point for Water

(Wet Point for Oil)

The wet point for oil was determined by the following protocol.
(1) 2 g of the powder component was kneaded with a spatula on a glass plate while adding isononyl isononanoate with a viscosity of 9 cP at 25° C. and a density of 0.853 g/ml.
(2) When the powder component became completely wet and started to form a paste, the weight of the added oil was determined as the weight of the wet point.
(3) The wet point for oil was calculated from the equation: The wet point for oil (ml/100 g)={(the weight of wet point)/2 g}×100/the density of oil.

(Wet Point for Water)

The wet point for water was determined by the following protocol.
(1) 2 g of the powder component was kneaded with a spatula on a glass plate while adding water with a density of 0.998 g/ml.
(2) When the powder component became completely wet and started to form a paste, the weight of the added water was determined as the weight of the wet point.
(3) The wet point for water was calculated from the equation: The wet point for water (ml/100 g)={(the weight of the wet point)/2 g}×100/the density of water.

[Evaluation]

(Squalene Oxidation Inhibition Evaluation)

Benzopyrene was used as a representative of a pollutant. Benzopyrene is known as a substance which promotes the oxidation of sebum. Squalene was used as a representative of sebum.

Each of the above compositions according to Example 1 and Comparative Examples 1-4 was mixed with a dispersion including benzopyrene (an aqueous dispersion prepared by mixing in water 1% by weight of benzopyrene, 5% by weight of acetone, and 10% by weight of polyglyceryl-10 laurate) in the same manner to obtain a mixture.

5 g of the obtained mixture was dried at 50° C. for one night. 5 g of water was added to the dried mixture, and the dried mixture was re-dispersed by hand shaking. Squalene was added to the dispersion such that the concentration of squalene was 0.01% by weight. The obtained dispersion was irradiated with UV rays with Suntest CPS (TOYO SEIKI, 765 W/m²) for 15 minutes. The level of hydroperoxide was analyzed with an LPO kit (Lipid Hydroperoxide Assay kit from Cayman).

The results are shown in the “Hydroperoxide Concentration (μM)” line in Table 1.

(Results)

The test results in Table 1 show that the anti-oxidation effects of the composition according to Example 1 are higher than those of the composition according to Comparative Examples 1-4.

The comparison of the anti-oxidation effects of the composition according to Example 1 and those of the composition according to Comparative Example 1 demonstrates that a combination of cellulose beads and a lipophilic antioxidant can provide excellent anti-oxidation effects.

The comparison of the anti-oxidation effects of the composition according to Example 1 and those of the composition according to Comparative Example 2 demonstrates that a combination of cellulose beads and a lipophilic antioxidant can provide better anti-oxidation effects than the single use of a lipophilic antioxidant.

The comparison of the anti-oxidation effects of the composition according to Example 1 and those of the composition according to Comparative Examples 3 and 4 demonstrates that a combination of cellulose beads and a lipophilic antioxidant can provide better anti-oxidation effects than a combination of cellulose beads and a hydrophilic antioxidant such as glutathione and ascorbic acid.

### Example 2 and Comparative Examples 5-6

[Preparation]

The following cosmetic compositions according to Example 2 and Comparative Examples 5-6, shown in Table 3, were prepared by mixing the ingredients shown in Table 3. The numerical values for the amounts of the ingredients shown in Table 3 are all based on “% by weight” as active raw materials. The cellulose beads in Table 3 were the same as those used in Table 1.

TABLE 3

| | | Comp. | Comp. |
|---|---|---|---|
| | Ex. 2 | Ex. 5 | Ex. 6 |
| Form | W/O emulsion | | |
| Tocopherol | 0.05 | — | 0.05 |
| Cellulose Beads | 2 | — | — |
| Disodium EDTA | 0.1 | 0.1 | 0.1 |
| Sodium Chloride | 0.1 | 0.1 | 0.1 |
| Dicaprylyl Carbonate | 31.25 | 31.25 | 31.25 |
| Polyglyceryl-6 Polyricinoleate | 0.5 | 0.5 | 0.5 |
| PEG-32 | 0.5 | 0.5 | 0.5 |
| Phenoxyethanol | 0.7 | 0.7 | 0.7 |
| Dimethicone | 10 | 10 | 10 |
| Water | qsp 100 | qsp 100 | qsp 100 |
| Propylene Glycol | 5 | 5 | 5 |
| Pentylene Glycol | 1 | 1 | 1 |
| Caprylyl Glycol | 0.5 | 0.5 | 0.5 |
| Hydroperoxide | 15 | 20 | 40 |
| Concentration (μM) | | | |

[Evaluation]

(Squalene Oxidation Inhibition Evaluation)

Benzopyrene was used as a representative of a pollutant. Squalene was used as a representative of sebum.

Each of the above compositions according to Example 2 and Comparative Examples 5-6 was mixed with a dispersion including benzopyrene (an aqueous dispersion prepared by mixing in water 1% by weight of benzopyrene, 5% by weight of acetone, and 10% by weight of polyglyceryl-10 laurate) in the same manner to obtain a mixture.

5 g of the obtained mixture was dried at 50° C. for one night. 5 g of water was added to the dried mixture, and the dried mixture was re-dispersed by hand shaking. Squalene was added to the dispersion such that the concentration of squalene was 0.01% by weight. The obtained dispersion was irradiated with UV rays with Suntest CPS (TOYO SEIKI, 765 W/m²) for 15 minutes. The level of hydroperoxide was analyzed with an LPO kit (Lipid Hydroperoxide Assay kit from Cayman).

The results are shown in the “Hydroperoxide Concentration (μM)” line in Table 3.

(Results)

The test results in Table 3 show that the anti-oxidation effects of the composition according to Example 2 are higher than those of the composition according to Comparative Examples 5-6.

The comparison of the anti-oxidation effects of the composition according to Example 2 and those of the composition according to Comparative Example 5 demonstrates that a combination of cellulose beads and a lipophilic antioxidant can provide excellent anti-oxidation effects.

The comparison of the anti-oxidation effects of the composition according to Example 1 and those of the composition according to Comparative Example 6 demonstrates that a combination of cellulose beads and a lipophilic antioxidant can provide better anti-oxidation effects than the single use of a lipophilic antioxidant.

The comparison of the anti-oxidation effects of the composition according to Example 1 in Table 1 and those of the composition according to Example 2 in Table 3 show that the present invention in the form of a two-phase formulation can provide better anti-oxidation effects than the present invention in the form of an W/O emulsion.

1. A composition comprising:
(a) at least one particle having
a wet point for oil being at least 100 ml/100 g, and
a wet point for water being at least 100 ml/100 g; and
(b) at least one lipophilic antioxidant.

2. The composition according to claim 1, wherein the number-average primary particle size of the (a) particle is 50 μm or less.

3. The composition according to claim 1, wherein the ratio of the wet point for water/the wet point for oil of the (a) particle is 5 or less.

4. The composition according to claim 1, wherein the (a) particle is porous.

5. The composition according to claim 1, wherein the (a) particle comprises at least one material selected from the group consisting of polysaccharides, silicon compounds, boron compounds, metal compounds, polymers, perlites, and mixtures thereof.

6. The composition according to claim 1, wherein the (a) particle comprises at least one polysaccharide.

7. The composition according to claim 1, wherein the amount of the (a) particle in the composition is from 0.01% to 20% by weight relative to the total weight of the composition.

8. The composition according to claim 1, wherein the (b) lipophilic antioxidant agent is selected from the group consisting of pentaerythrityl tetra-di-t-butyl hydroxyhydrocinnamate, nordihydroguaiaretic acid, propyl gallate, butylated hydroxytoluene, butylated hydroxyanisole, ascorbyl palmitate, tocopherol and mixtures thereof.

9. The composition according to claim 1, wherein the (b) lipophilic antioxidant agent is at least one tocopherol selected from the group consisting of α-tocopherol, β-tocopherol, γ-tocopherol, δ-tocopherol and a mixture thereof.

10. The composition according to claim 1, wherein the amount of the (b) lipophilic antioxidant agent in the composition is from 0.001% to 5% by weight relative to the total weight of the composition.

11. The composition according to claim 1, wherein the composition further comprises (c) at least one oil or (d) water.

12. The composition according to claim 1, wherein the composition further comprises (c) at least one oil and (d) water.

13. The method according to claim 14, wherein the method is intended for protecting skin from damage selected from the group consisting of oily skin, dehydration of skin, alteration of desquamation, squalene decrease, vitamin E decrease, pigmentation, pore problems such as clogged pores, dilated pores, acne and black heads, loss of dry/oily balance, dull skin, aging, and lactic acid increase.

14. A non-therapeutic method comprising: applying onto the keratin materials a composition comprising:
(a) at least one particle having
a wet point for oil being at least 100 ml/100 g, and
a wet point for water being at least 100 ml/100 g; and
(b) at least one lipophilic antioxidant agent.

15. A method of using a combination of
(a) at least one particle having
a wet point for oil being at least 100 ml/100 g, and
a wet point for water being at least 100 ml/100 g; and
(b) at least one lipophilic antioxidant agent for reducing or controlling oxidation of unsaturated lipids.
